(19) United States
(12) Patent Application Publication
(10) Pub. No.: US 2018/0184927 A1
(43) Pub. Date: Jul. 5, 2018

(54) REAL-TIME HEART RATE DETECTION METHOD AND REAL-TIME HEART RATE DETECTION SYSTEM THEREFOR

(71) Applicant: EOSMEM Corporation, Zhubei City (TW)

(72) Inventors: Chu-Hsin Chang, Zhubei City (TW); Jun-Shian Hsiao, Chupei (TW); Ching-Lung Ti, Chupei (TW); Chun-Fu Lin, Chupei (TW); Hui-Min Tsai, Taipei (TW)

(21) Appl. No.: 15/858,782

(22) Filed: Dec. 29, 2017

Related U.S. Application Data
(60) Provisional application No. 62/440,746, filed on Dec. 30, 2016.

(30) Foreign Application Priority Data
Jul. 20, 2017 (TW) 106124209

(51) Int. Cl. A61B 5/024; A61B 5/00

(52) U.S. Cl. CPC A61B 5/02416; G06K 9/00013; A61B 5/725

(57) ABSTRACT

A real-time heart rate detection method for use in a real-time heart rate detection system includes: (A) emitting light to a finger to generate reflected light; (B) receiving the reflected light via a sensing unit, to generate at least one initial fingerprint image; (C) generating plural initial waveform information according to the at least one initial fingerprint image; (D) selecting one among plural different bandpass filters, to filter the initial waveform information; (E) calculating an initial heart rate based upon the filtered initial waveform information; (F) checking and computing a frequency range of the obtained initial heart rate, to determine which one of the plural bandpass filters is the most preferable bandpass filter; (G) outputting a final heart rate; and repeating the step (A) to the step (G). The step (G) and the step (F) are performed at least partially in parallel.

### CROSS REFERENCE

The present invention claims priority to U.S. 62/440,746, filed on Dec. 30, 2016 and claims priority to TW 106124209 filed on Jul. 20, 2017.

### BACKGROUND OF THE INVENTION

### Field of Invention

The present invention relates to a real-time heart rate detection method and a real-time heart rate detection system therefor; particularly, it relates to such real-time heart rate detection method and real-time heart rate detection system capable of reporting a current heart rate as well as a long-term stable heart rate in real-time by switching among different bandpass filters at a most appropriate time point through a multi-thread parallel processing method.

### Description of Related Art

A conventional fingerprint identifier can only identify fingerprint patterns but does not possess any other function.

There are prior art proposing to detect heart rate through image identification technique. However, so far there is no prior art which integrates fingerprint identification capability and heart rate detection capability in one device.

In addition, among the prior art heart rate detection methods through image identification technique, if a more precise (i.e., a narrower frequency band) bandpass filter is adopted in detecting heart rate, there is a likelihood that the heart rate might not fall within this narrow frequency band (for example, for a same person, his or her heart rate are different while he or she is doing exercise or sleeping). On the other hand, if a bandpass filter having a broader frequency band is adopted, the accuracy for heart rate detection could be potentially compromised. For at least the above reason, the real-time heart rate detection accuracy of the prior art heart rate detection methods is not satisfactory.

In view of the above, to overcome the drawback in the prior art, the present invention proposes a real-time heart rate detection method and a real-time heart rate detection system capable of reporting a current heart rate as well as a long-term stable heart rate in real-time by switching among different bandpass filters at a most appropriate time point through a multi-thread parallel processing method.

### SUMMARY OF THE INVENTION

From one perspective, the present invention provides a real-time heart rate detection method for detecting a real-time heart rate through sensing a feature of a finger, the real-time heart rate detection method comprising the steps of: (A) emitting light to the finger to generate reflected light; (B) receiving the reflected light via a sensing unit, to generate at least one initial fingerprint image; (C) generating plural initial waveform information according to the at least one initial fingerprint image; (D) selecting one among plural different bandpass filters, to filter the initial waveform information; (E) calculating an initial heart rate based upon the filtered initial waveform information; (F) checking and computing a frequency range of the obtained initial heart rate, to determine which one of the plural bandpass filters is the most preferable bandpass filter; (G) outputting a final heart rate; and repeating the step (A) to the step (G), wherein while repeating the step (D) in a current iteration, selecting one of the bandpass filters according to a result from the step (F) in a previous iteration; wherein, the step (G) and the step (F) are performed at least partially in parallel; and wherein, the step (A) to step (C) in the current iteration and the step (F) to step (G) in the previous iteration are performed at least partially in parallel.

In one embodiment, the step (D) comprises the steps of: (D1) selecting one among the plural different bandpass filters, to filter a noise of the plural initial waveform information, thereby generating a pre-processed waveform information; (D2) performing low pass filtering to the pre-processed waveform information, to obtain an average of the pre-processed waveform information, thus generating averaged waveform information; and (D3) generating period information according to intersecting points where the pre-processed waveform information and the averaged waveform information intersects with each other.

In one embodiment, the step (E) comprises: generating the initial heart rate according to the period information.

In one embodiment, the step (F) comprises the steps of: (F1) checking whether or not the initial heart rate is a stable constant; when yes, proceeding to the step (F2); and when no, proceeding to the step (F3); (F2) determining which one of the plural bandpass filters is the most preferable bandpass filter according to the stable constant and subsequently proceeding to the step (F3); and (F3) performing low pass filtering to the initial heart rate, to output the final heart rate.

From another perspective, the present invention provides a

real-time heart rate detection system for detecting a real-time heart rate through sensing a feature of a finger, the real-time heart rate detection system comprising: a fingerprint sensor including a light source and a sensing unit, wherein the light source is configured to operably emit light to the finger to generate reflected light, and wherein the sensing unit is configured to operably receive the reflected light, to generate at least one initial fingerprint image; a pixel information receiver configured to operably generate plural initial waveform information according to the at least one initial fingerprint image; and a computing unit including plural different bandpass filters, wherein the computing unit selects one among plural different bandpass filters, to filter the initial waveform information, and wherein the computing unit calculates an initial heart rate based upon the filtered initial waveform information; wherein, the computing unit is configured to operably check and compute a frequency range of the obtained initial heart rate, to determine which one of the plural bandpass filters is the most preferable bandpass filter; and wherein, the computing unit is configured to operably output a final heart rate; wherein, that the computing unit checks and computes a frequency range of the obtained initial heart rate to determine which one of the plural bandpass filters is the most preferable bandpass filter and that the computing unit outputs a final heart rate are performed at least partially in parallel.

In one embodiment, the computing unit includes: a waveform filtering unit, configured to operably generate a period information according to the plural initial waveform information; a heart rate calculating unit, configured to operably generate an initial heart rate according to the period information; and a checking unit, configured to operably check which frequency band the obtained initial heart rate falls within, to determine which one of the plural bandpass filters is the most preferable bandpass filter and check whether or not the initial heart rate is stable.

In one embodiment, the waveform filtering unit includes: a multi-thread bandpass filter including plural different bandpass filters, wherein the plural different bandpass filters are configured to operably filter a noise of the plural initial waveform information, thereby generating pre-processed waveform information; and a low-pass filter, configured to operably perform low pass filtering to the pre-processed waveform information, to obtain an average of the pre-processed waveform information, thus generating an averaged waveform information; wherein, the waveform filtering unit generates the period information according to intersecting points where the pre-processed waveform information and the averaged waveform information intersects with each other.

In one embodiment, when the initial heart rate is stable, the checking unit outputs a frequency band switching signal to the pixel information receiver, for selecting one bandpass filter among plural different bandpass filters.

In one embodiment, when the initial heart rate is unstable, the checking unit performs low pass filtering to the initial heart rate, and outputting the final heart rate.

In one embodiment, the feature of the finger is a fingerprint pattern.

The objectives, technical details, features, and effects of the present invention will be better understood with regard to the detailed description of the embodiments below, with reference to the attached drawings.

### BRIEF DESCRIPTION OF THE DRAWINGS

FIG. 1 is a flowchart showing a real-time heart rate detection method according to an embodiment of the present invention.

FIG. 2 is a flowchart showing a real-time heart rate detection method according to a specific embodiment of the present invention.

FIG. 3 shows a schematic block diagram of an embodiment of the present invention, illustrating a real-time heart rate detection system adopting a real-time heart rate detection method according to the present invention.

FIG. 4 shows a schematic block diagram of an embodiment of a computing unit.

FIG. 5 shows a schematic block diagram of a specific embodiment of a computing unit.

FIG. 6 shows that, in the real-time heart rate detection method according to the present invention, the step (A) to step (C) in the current iteration and the step (F) to step (G) in the previous iteration can be performed at least partially in parallel.

FIG. 7 shows an example as to how period information which is related to a heart rate period is generated.

FIG. 8 shows that plural different bandpass filters have respective different frequency bands, respectively, which correspond to respective different heart rate frequency ranges.

FIG. 9 shows a Bode plot, illustrating a curve of frequency response in decibel (dB) versus frequency.

### DESCRIPTION OF THE PREFERRED EMBODIMENTS

The above and other technical details, features and effects of the present invention will be will be better understood with regard to the detailed description of the embodiments below, with reference to the drawings. The drawings as referred to throughout the description of the present invention are for illustration only, to show the interrelations between the steps or components, but not drawn according to actual scale.

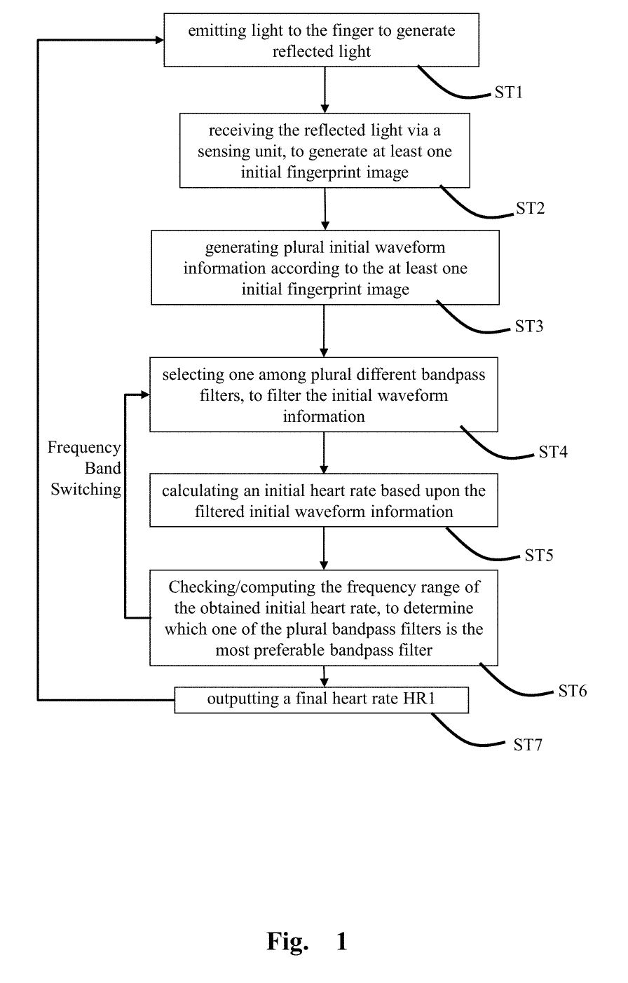

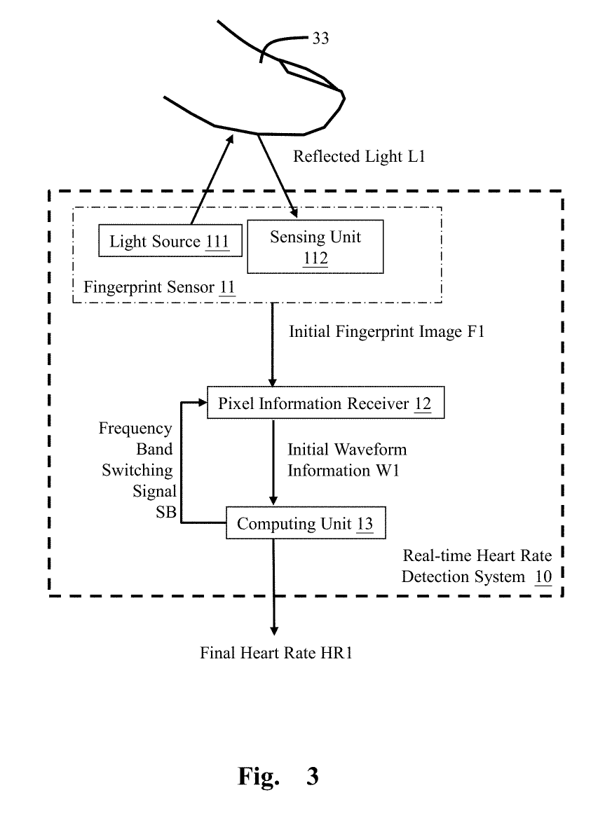

Please refer to FIG. 1 in conjugation with FIG. 3. FIG. 1 is a flowchart showing a real-time heart rate detection method according to an embodiment of the present invention. FIG. 3 shows a schematic block diagram of an embodiment of the present invention, illustrating a real-time heart rate detection system adopting a real-time heart rate detection method according to the present invention.

As shown in FIG. 3, the real-time heart rate detection system 10 of this embodiment can detect a real-time heart rate through sensing a feature of a finger 33. In one embodiment, the feature of the finger 33 can be, for example but not limited to, a fingerprint pattern.

The real-time heart rate detection system 10 comprises: a fingerprint sensor 11, a pixel information receiver 12 and a computing unit 13. The fingerprint sensor 11 includes light source 111 and a sensing unit 112. In one embodiment, the sensing unit 112 can be, for example but not limited to, a pixel array sensing unit. The light source 111 is configured to operably emit light to the finger 33 to generate reflected light L1 (referring to step ST1 in FIG. 1). The sensing unit 112 is configured to operably receive the reflected light L1, to generate at least one initial fingerprint image F1 (referring to step ST2 in FIG. 1).

The pixel information receiver 12 is configured to operably generate plural initial waveform information W1 according to the at least one initial fingerprint image F1 (referring to step ST3 in FIG. 1).

The computing unit 13 includes plural different bandpass filters 131A (referring to FIG. 5). In one embodiment, the computing unit 13 can select one among plural different bandpass filters 131A, to filter the initial waveform information W1 (referring to step ST4 in FIG. 1). And, the computing unit 13 can calculate an initial heart rate HR0 (referring to step ST5 in FIG. 1) based upon the filtered initial waveform information.

One of the features and advantages of the present invention is that: after the initial heart rate HR0 has been obtained, the computing unit 13 will further check the frequency range of initial heart rate HR0 obtained via the above-mentioned step ST5, to determine which one of the plural bandpass filters is the most preferable bandpass filter (referring to step ST6 in FIG. 1). As such, the present invention is capable of adopting a more precise (i.e., a narrower frequency band) bandpass filter, to obtain more accurate heart rate information.

After step ST6, the computing unit 13 will output a final heart rate HR1 (referring to step ST7 in FIG. 1).

To be more specific, the present invention has the following features and advantages:

First, after the final heart rate HR1 is outputted, the real-time heart rate detection system 10 of the present invention adopting the real-time heart rate detection method will repeat the steps ST1-ST7. While repeating the step ST4, the computing unit 13 will select one of the bandpass filter 131A according to the result from the step ST6 in the previous iteration (one iteration includes the steps ST1-ST7); that is, after the initial heart rate HR0 is obtained, the computing unit 13 will check the frequency range of the initial heart rate HR0, so as to determine which one of the plural bandpass filters is the most preferable bandpass filter.

Second, please refer to FIG. 1. As shown in FIG. 1, the step that the computing unit 13 checks the frequency range of the obtained initial heart rate HR0 to determine which one of the plural bandpass filters is the most preferable bandpass filter (i.e., the step ST6) and the step that the computing unit 13 outputs a final heart rate HR1 (i.e., the step ST7) are performed at least partially in parallel.

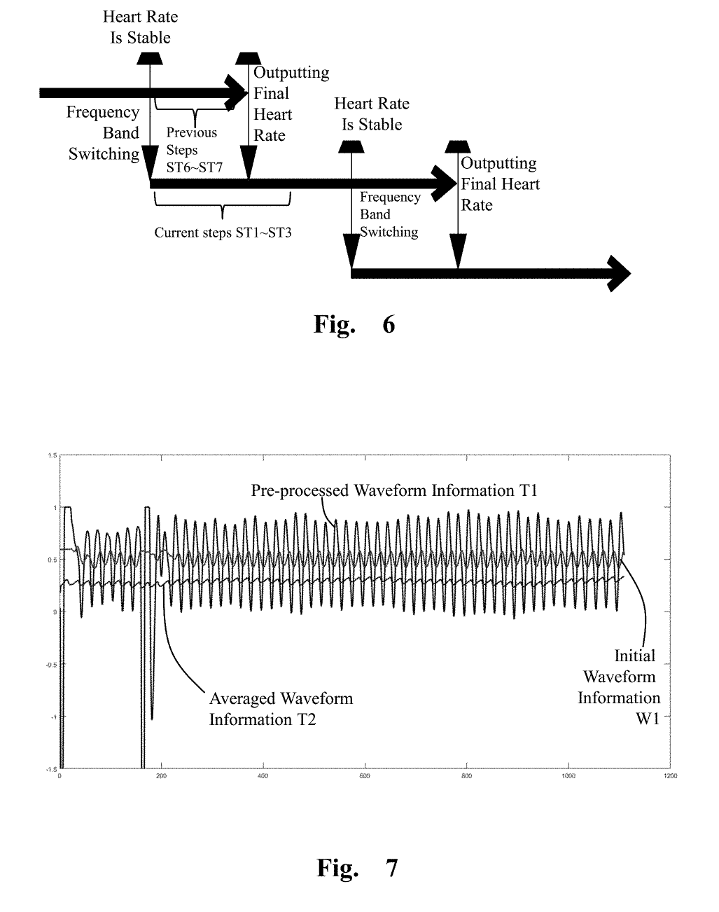

Third, please refer to FIG. 6. As shown in FIG. 6, the steps ST1 to ST3 in the current iteration and the steps ST6 to ST7 in the previous iteration are performed at least partially in parallel.

It is noteworthy that, because the step ST6 and the step ST7 are performed at least partially in parallel and because the steps ST1 to ST3 in the current iteration and the steps ST6 to ST7 in the previous iteration are performed at least partially in parallel, on the one hand, the present invention is able to switch among different bandpass filters to obtain the most accurate detection result, and on the other hand, the multi-thread parallel processing can shorten the processing time, so as to report the current heart rate as well as a long-term stable heart rate in real-time.

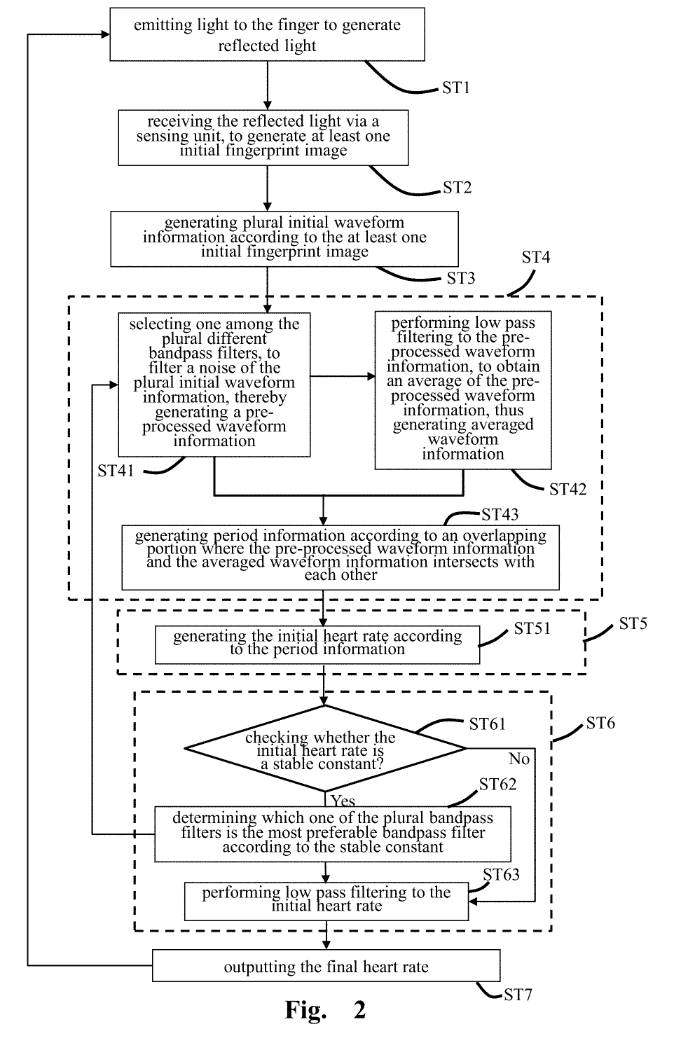

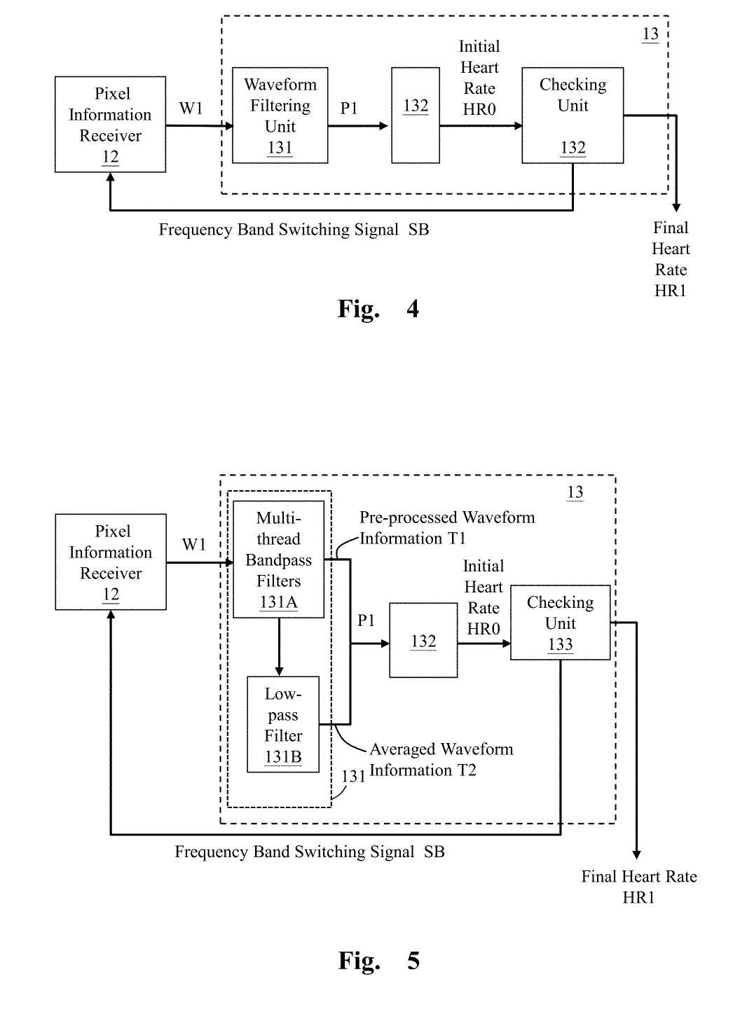

Please refer to FIG. 2 in conjugation with FIG. 4. FIG. 2 is a flowchart showing a real-time heart rate detection method according to a specific embodiment of the present invention. FIG. 4 shows a schematic block diagram of an embodiment of a computing unit.

As shown in FIG. 4, in one embodiment, the computing unit 13 includes, for example but not limited to: a waveform filtering unit 131, a heart rate calculating unit 132 and a checking unit 133.

The waveform filtering unit 131 is configured to operably generate period information P1 according to the plural initial waveform information W1 (referring to step ST4 in FIG. 2). The heart rate calculating unit 132 is configured to operably generate an initial heart rate HR0 according to the period information P1 (referring to step ST51 in FIG. 2). The checking unit 133, on one hand, is configured to operably check which frequency band the obtained initial heart rate HR0 falls within, to determine which one of the plural bandpass filters is the most preferable bandpass filter. Besides, on the other hand, the checking unit 133 can check whether or not the initial heart rate HR0 is a stable constant (referring to step ST61 in FIG. 2).

Please refer to FIG. 2 in conjugation with FIG. 5 and FIG. 7. FIG. 5 shows a schematic block diagram of a specific embodiment of a computing unit. FIG. 7 shows an example as to how period information which is related to a heart rate period is generated.

As shown in FIG. 5, in one embodiment, the waveform filtering unit 131 of the computing unit 13 includes, for example but not limited to: a multi-thread bandpass filter 131A and a low-pass filter 131B.

The multi-thread bandpass filter 131A includes plural different bandpass filters. The multi-thread bandpass filter 131A is configured to operably filter a noise of the plural initial waveform information W1, to generate pre-processed waveform information T1 (as shown in FIG. 7; also, referring to step ST41 in FIG. 2).

It is noteworthy that another feature and advantage of the present invention is that: because the multi-thread bandpass filter 131A includes plural different bandpass filters, the waveform filtering unit 131 of the computing unit 13 can select one of the plural different bandpass filters of the multi-thread bandpass filter 131A according to a result from the step ST6 in the previous iteration (that is, after the initial heart rate HR0 is obtained, the waveform filtering unit 131 of the computing unit 13 will further check and compute a frequency range of initial heart rate HR0 obtained via step ST5, to determine which one of the plural bandpass filters of the multi-thread bandpass filter 131A is the most preferable bandpass filter). As a result, after repeating t serval iterations, the noise in the pre-processed waveform information T1 will be greatly reduced.

The low-pass filter 131B is configured to operably perform low pass filtering to the pre-processed waveform information T1, to obtain an average of the pre-processed waveform information T1, thus generating averaged waveform information T2 (as shown in FIG. 7; also, referring to step ST42 in FIG. 2).

Next, the waveform filtering unit 131 generates period information P1 according to i where the pre-processed waveform information T1 and the averaged waveform information T2 intersects with each other (as shown in FIG. 5 and FIG. 7; also, referring to step ST43 in FIG. 2).

Please refer to FIG. 2 in conjugation with FIG. 4 and FIG. 5. It is noteworthy that yet another feature and advantage of the present invention is that: the checking unit 133, on one hand, is configured to operably check which frequency band the obtained initial heart rate HR0 falls within, to determine which one of the plural bandpass filters is the most preferable bandpass filter, and on the other hand, the checking unit 133 can check whether or not the initial heart rate HR0 is a stable constant (referring to step ST61 in FIG. 2).

When the initial heart rate HR0 is a stable constant, the checking unit 133 outputs a frequency band switching signal SB to the pixel information receiver 12, to select one among plural different bandpass filters based upon this stable constant (referring to step ST62 in FIG. 2). While repeating the step ST4 in the next iteration, one of the bandpass filters is selected according to a result from the step ST6 in the previous iteration.

Besides, preferably, in one embodiment, the checking unit 133 can perform low pass filtering to the initial heart rate HR0 (referring to step ST63 in FIG. 2).

When the initial heart rate HR0 is nota stable constant, after the step ST61, the present invention will directly proceed to the step ST63. And, next, the checking unit 133 will output the final heart rate HR1 (referring to step ST7 in FIG. 2).

It is noteworthy that: the above-mentioned steps “when the initial heart rate HR0 is a stable constant, the checking unit 133 outputs a frequency band switching signal SB to the pixel information receiver 12, to select one among plural different bandpass filters based upon such stable constant (referring to step ST62 in FIG. 2). While repeating the step ST4 in the next iteration, one of the bandpass filters is selected according to a result from the step ST6 in the previous iteration” and the above-mentioned steps “the checking unit 133 performs low pass filtering to the initial heart rate HR0 (referring to step ST63 in FIG. 2). And, next, the checking unit 133 will output the final heart rate HR1 (referring to step ST7 in FIG. 2)” are performed at least partially in parallel. As such, the present invention can report a current heart rate as well as a long-term stable heart rate in real-time by switching among different bandpass filters at a most appropriate time point through multi-thread parallel processing.

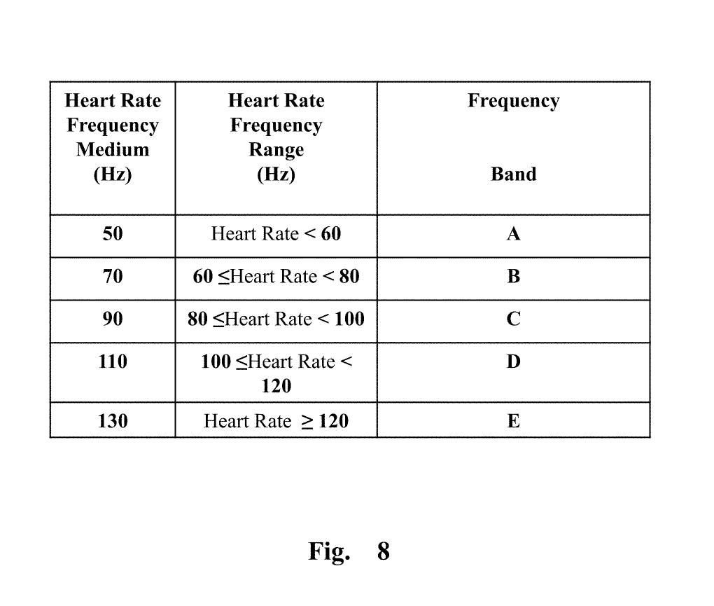

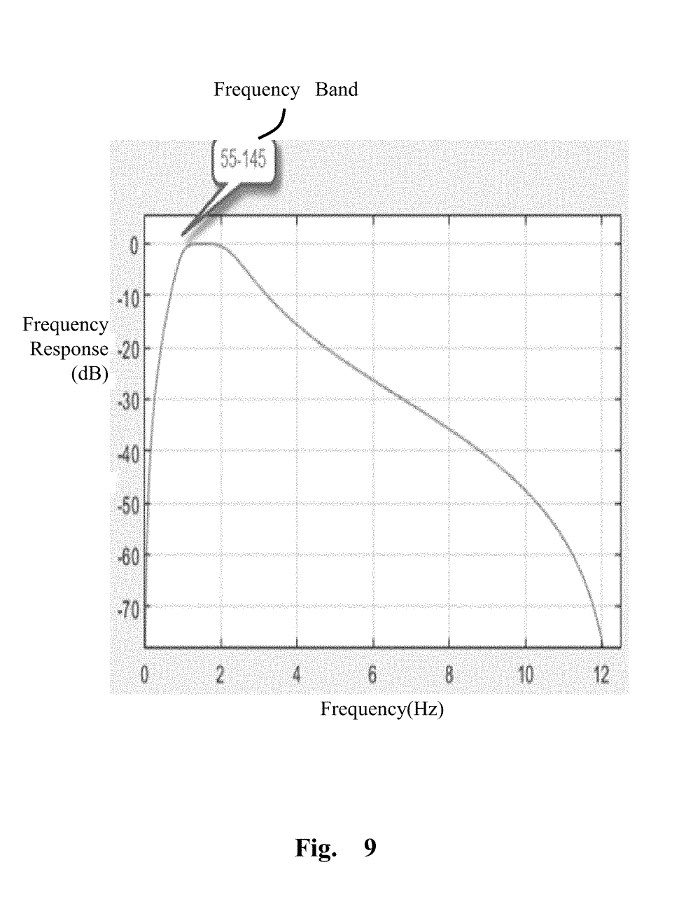

For the details as to how the present invention reports current heart rate as well as a long-term stable heart rate in real-time by switching among different bandpass filters at a most appropriate time point through a multi-thread parallel processing method, please refer to FIGS. 6, 8 and 9. FIG. 8 shows that plural different bandpass filters have respective different frequency bands, which correspond to respective different heart rate frequency ranges. FIG. 9 shows a Bode plot, illustrating a curve of frequency response in decibel (dB) versus frequency.

As shown in FIG. 8, different individuals have different respective heart rate frequency ranges. One advantage of the present invention is that: providing different frequency bands in correspondence to different heart rate frequency ranges. The above-mentioned multi-thread bandpass filter 131A includes plural different bandpass filters, and different bandpass filters have different frequency bands. As shown in FIG. 8, for example, when an individual has a heart rate frequency range which is smaller than 60 Hz, the corresponding frequency band will be A. But, when another individual has a heart rate frequency range which is between 80 Hz-100 Hz, the corresponding frequency band will be C. In one embodiment, the frequency bands A-E as shown in FIG. 8 are non-overlapped with one another. In another embodiment, the frequency bands A-E can be overlapped with one another. Certainly, the number and ranges of the bandpass filters are not limited to the way shown in FIG. 8 but can be modified as desired.

For example, in one embodiment, while filtering a noise of the plural initial waveform information W1 for a first time, a frequency band of a selected bandpass filter can be, for example but not limited to, 36 Hz-180 Hz. Note that such frequency band of 36 Hz-180 Hz is wider than the ranges of all the above-mentioned frequency bands A-E. Next, after the real-time heart rate detection method of the present invention repeats the steps ST1-ST7 in several iterations (referring to FIG. 1) and finds that the initial heart rate is a stable constant, the present invention will then switch the frequency band of 36 Hz-180 Hz to another frequency band (referring also to FIG. 6). For example, assuming that an individual has a heart rate frequency range which is between 80 Hz-100 Hz, then, in step ST4, the bandpass filter having a corresponding frequency band of C among the plural different bandpass filters, is selected (referring also to FIG. 6).

Because the present invention adopts a multi-thread bandpass filter which is able to dynamically switch among different bandpass filters, the present invention is capable of obtaining a most accurate heart rate in response to not only different individuals having different heart rate characteristics but also different individuals in different physiological statuses.

The present invention has been described in considerable detail with reference to certain preferred embodiments thereof. It should be understood that the description is for illustrative purpose, not for limiting the scope of the present invention. An embodiment or a claim of the present invention does not need to achieve both the objectives or advantages of the present invention. The title and abstract are provided for assisting searches but not for limiting the scope of the present invention. Those skilled in this art can readily conceive variations and modifications within the spirit of the present invention. It is not limited for each of the embodiments described hereinbefore to be used alone; under the spirit of the present invention, two or more of the embodiments described hereinbefore can be used in combination. For example, two or more of the embodiments can be used together, or, a part of one embodiment can be used to replace a corresponding part of another embodiment. In view of the foregoing, the spirit of the present invention should cover both such and other modifications and variations, which should be interpreted to fall within the scope of the following claims and their equivalents.

What is claimed is:

1. A real-time heart rate detection method for detecting a real-time heart rate through sensing a feature of a finger, the real-time heart rate detection method comprising the steps of:
(A) emitting light to the finger to generate reflected light;
(B) receiving the reflected light via a sensing unit, to generate at least one initial fingerprint image;
(C) generating plural initial waveform information according to the at least one initial fingerprint image;
(D) selecting one among plural different bandpass filters, to filter the initial waveform information;
(E) calculating an initial heart rate based upon the filtered initial waveform information;
(F) checking and computing a frequency range of the obtained initial heart rate, to determine which one of the plural bandpass filters is the most preferable bandpass filter;
(G) outputting a final heart rate; and
repeating the step (A) to the step (G), wherein while repeating the step (D) in a current iteration, selecting one of the bandpass filters according to a result from the step (F) in a previous iteration;
wherein, the step (G) and the step (F) are performed at least partially in parallel; and
wherein, the step (A) to step (C) in the current iteration and the step (F) to step (G) in the previous iteration are performed at least partially in parallel.

2. The real-time heart rate detection method of claim 1, wherein the step (D) comprises the steps of:
(D1) selecting one among the plural different bandpass filters, to filter a noise of the plural initial waveform information, thereby generating a pre-processed waveform information;
(D2) performing low pass filtering to the pre-processed waveform information, to obtain an average of the pre-processed waveform information, thus generating averaged waveform information; and
(D3) generating period information according to intersecting points where the pre-processed waveform information and the averaged waveform information intersects with each other.

3. The real-time heart rate detection method of claim 2, wherein the step (E) comprises: generating the initial heart rate according to the period information.

4. The real-time heart rate detection method of claim 2, wherein the step (F) comprises the steps of:
(F1) checking whether or not the initial heart rate is a stable constant; when yes, proceeding to the step (F2); and
when no, proceeding to the step (F3);
(F2) determining which one of the plural bandpass filters is the most preferable bandpass filter according to the stable constant and subsequently proceeding to the step (F3); and
(F3) performing low pass filtering to the initial heart rate, to output the final heart rate.

5. The real-time heart rate detection method of claim 1, wherein the feature of the finger is a fingerprint pattern.

6. A real-time heart rate detection system for detecting a real-time heart rate through sensing a feature of a finger, the real-time heart rate detection system comprising:
a fingerprint sensor including a light source and a sensing unit, wherein the light source is configured to operably emit light to the finger to generate reflected light, and wherein the sensing unit is configured to operably receive the reflected light, to generate at least one initial fingerprint image;
a pixel information receiver configured to operably generate plural initial waveform information according to the at least one initial fingerprint image; and
a computing unit including plural different bandpass filters, wherein the computing unit selects one among plural different bandpass filters, to filter the initial waveform information, and wherein the computing unit calculates an initial heart rate based upon the filtered initial waveform information;
wherein, the computing unit is configured to operably check and compute a frequency range of the obtained initial heart rate, to determine which one of the plural bandpass filters is the most preferable bandpass filter; and
wherein, the computing unit is configured to operably output a final heart rate;
wherein, that the computing unit checks and computes a frequency range of the obtained initial heart rate to determine which one of the plural bandpass filters is the most preferable bandpass filter and that the computing unit outputs a final heart rate are performed at least partially in parallel.

7. The real-time heart rate detection system of claim 6, wherein the computing unit includes:
a waveform filtering unit, configured to operably generate period information according to the plural initial waveform information;
a heart rate calculating unit, configured to operably generate an initial heart rate according to the period information; and
a checking unit, configured to operably check which frequency band the obtained initial heart rate falls within, to determine which one of the plural bandpass filters is the most preferable bandpass filter and check whether or not the initial heart rate is stable.

8. The real-time heart rate detection system of claim 7, wherein the waveform filtering unit includes:
a multi-thread bandpass filter including plural different bandpass filters, wherein the plural different bandpass filters are configured to operably filter a noise of the plural initial waveform information, thereby generating pre-processed waveform information; and
a low-pass filter, configured to operably perform low pass filtering to the pre-processed waveform information, to obtain an average of the pre-processed waveform information, thus generating an averaged waveform information;
wherein, the waveform filtering unit generates the period information according to intersecting points where the pre-processed waveform information and the averaged waveform information intersects with each other.

9. The real-time heart rate detection system of claim 7, wherein when the initial heart rate is stable, the checking unit outputs a frequency band switching signal to the pixel information receiver, for selecting one bandpass filter among plural different bandpass filters.

10. The real-time heart rate detection system of claim 7, wherein when the initial heart rate is unstable, the checking unit performs low pass filtering to the initial heart rate, and outputting the final heart rate.

11. The real-time heart rate detection system of claim 7, wherein the feature of the finger is a fingerprint pattern.
